(19) United States
(12) Patent Application Publication
(10) Pub. No.: US 2004/0132751 A1
(43) Pub. Date: Jul. 8, 2004

(54) Use of desoxypeganine for treating central nervous system symptoms resulting from intoxications by psychotrops

(76) Inventors: Klaus Opitz, Muenster (DE); Joachim Moormann, Werne (DE); Thomas Hille, Neuwied (DE); Frank Becher, Koblenz (DE)

Correspondence Address: BIRCH STEWART KOLASCH & BIRCH, PO BOX 747, FALLS CHURCH, VA, 22040-0747, US

(21) Appl. No.: 10/475,788

(22) PCT Filed: Apr. 18, 2002
(86) PCT No.: PCT/EP02/04278

(30) Foreign Application Priority Data
Apr. 24, 2001 (DE) 101 19 863.9

(51) Int. Cl. A61K 31/519

(52) U.S. Cl. 514/267

(57) ABSTRACT

The invention relates to the use of deoxypeganine, as free base or as acid addition salt, for the treatment of cerebral, central nervous or psychiatric symptoms, defunctionalization manifestations or disorders occurring through intake of psychotropic substances as a consequence of occasional or chronic abuse of addictive substances, intoxicants or medicines, or as side effects of the use, especially repeated or prolonged, as intended of medicaments, or as an effect of use, in particular repeated or prolonged, not as intended of medicaments, or as a result of acute poisoning by psychotropic toxic substances, or as a result of chronic exposure to toxic substances with a psychotropic effect in humans or other vertebrates.

The invention relates to the use of deoxypeganine for the treatment of disorders of the central nervous system such as cerebral, central nervous or psychiatric symptoms, defunctionalization manifestations or disorders which occur as a result of unintentional or intentional intake of psychotropic and/or hallucinogenic agents, e.g. environmental poisons, use and abuse of intoxicants or addictive substances, use and abuse of medicines, especially when there is dependence on addictive substances, especially alcohol dependence, in humans or in vertebrates.

Intake of psychotropic substances, including intoxicants, especially alcohol, is well known to lead to symptoms such as perception disturbances, memory loss, impairment of cognitive ability, general loss of control, aggressiveness, impairment of muscular coordination, etc.

If the substance is deliberately taken as intoxicant, such as, for example, heroin, cocaine or as ethyl alcohol-containing beverage, then although such effects are intended by the intoxicant-consuming person, they are also under certain conditions felt to be disadvantageous. An additional factor is that the severity and the duration of these symptoms may vary and is often difficult for the consumer of the intoxicant to estimate beforehand.

Especially when there is chronic dependence and continued abuse of addictive substances there is not only the generally known organic damage but there is also the occurrence of permanent defunctionalization manifestations which impair, for example, cognitive performance, especially memory performance. There may also be chronic manifestations of the previously mentioned psychiatric symptoms such as, for example, a general loss of control. These chronic sequelae of alcohol abuse—which occur in a similar way in other addictive substance dependences—represent a considerable impediment to successful implementation of detoxification therapies. Thus, it is known that the loss of control caused by chronic alcohol abuse makes abstinence impossible for the person affected by alcoholism. This is the main reason why even detoxified alcoholics are prone to relapses, usually with serious consequences. The principle that “controlled drinking” is impossible for dependent people was derived from this observation.

It is additionally known that there are great individual differences in intoxicant consumption behaviour, which is why, for example, alcoholics are divided into different categories of drinkers.

The problem for certain alcohol consumers is that, after a particular individual threshold dose has been exceeded, there is a rapid general loss of control with the abovementioned adverse side effects. The affected persons are usually unable to recognize in good time that they have reached their individual threshold dose or even their personal risk of relapse. The loss of control brought about thereby, which often leads to further excessive alcohol consumption, is the reason why such people are often referred to as dangerous drinkers. These are frequently people who have already undergone withdrawal therapies and relapse in this way.

It is known that the loss of control caused by chronic abuse of addictive substances, as well as the impairment of memory performance (and even dementia), often has far-reaching consequences for the affected person and for his surroundings, such as, for example, inability to carry on an occupation, inability to organize daily activities, inability to initiate and maintain social contacts and, resulting therefrom, social isolation.

These defunctionalization manifestations, e.g. the impairment of cognitive performance, often persist even after successfully completed withdrawal therapy. Further psychiatric or cerebral disturbances occurring in association with alcohol abuse or abuse of other addictive substances are, for example: perceptual illusions or hallucinations, amnesia, alterations of consciousness, formal cognitive disturbances, memory deficits, delusions, confabulations, disorientation, states of agitation.

The object therefore was to eliminate or at least alleviate the psychiatric symptoms or symptoms with a central nervous causation, occurring as a consequence of chronic abuse of addictive substances, in particular alcohol abuse, in particular the loss of control, loss of cognitive abilities, dementia, etc.

To solve this problem, the invention proposes using the agent deoxypeganine for the treatment of people suffering from such sequelae of dependencies on addictive substances. It is possible by administration of deoxypeganine to at least partially abolish or reverse the psychiatric or cerebral pathological manifestation caused as a result of chronic alcohol or intoxicant consumption, in particular the loss of cognitive abilities or loss of control occurring, so that the said abilities are gradually recovered. It is thus possible according to the present invention to eliminate or at least alleviate certain chronic symptoms of dependences on addictive substances.

The invention is based on the surprising observation that in animal experiments administration of deoxypeganine to rats was able to bring about a recovery of cognitive abilities. This restoration did not occur, or occurred only considerably later, in untreated control animals.

Deoxypeganine (1,2,3,9-tetrahydropyrrolo[2,1-b]quinazoline) is an alkaloid of molecular formula C₁₁H₁₂N₂ which is present in plants of the Zygophyllaceae family. Deoxypeganine is preferably obtained by isolation from Syrian rue (Peganum harmala) or by synthesis.

On the basis of its pharmacological properties, deoxypeganine is included in the group of reversibly acting cholinesterase inhibitors. It also acts as monoamine oxidase inhibitor.

Concerning use in medical therapy, it has been proposed to employ deoxypeganine for the treatment of Alzheimer's dementia, for the treatment of alcoholism through suppression of the desire for alcohol, for the treatment of nicotine dependence through reducing the desire for nicotine or for replacement therapy of drug addicts and for the treatment of withdrawal symptoms during withdrawal therapy. In addition, deoxypeganine can be employed as cholinesterase inhibitor as antidote or prophylactic in case of poisoning by organic phosphates, in which case it antagonizes the cerebral effect of cholinergic poisons.

According to the invention, deoxypeganine can be used both in the form of its free base and as acid addition salt for the treatment; preferred salts are deoxypeganine hydrochloride and deoxypeganine hydrobromide. It is also possible in addition to use salts of other pharmacologically acceptable acids, e.g. citrate, tartrate or acetate.

Deoxypeganine is preferably administered in a pharmaceutical preparation which contains the agent in proportions of from 0.1 to 90% by weight, particularly preferably in proportions of from 2 to 20% by weight, in each case calculated as free deoxypeganine. The deoxypeganine-containing pharmaceutical preparations used according to the invention may additionally contain excipients, carriers, stabilizers, etc., in the amounts known to the skilled person.

The dose administered each day is preferably in the range from 0.1 to 100 mg, in particular from 10 to 50 mg. It should be adjusted appropriately depending on the individual requirements.

The preparations which are used according to the present invention for administering deoxypeganine may contain one or more of the following additives:

antioxidants, synergists, stabilizers;

preservatives;

taste masking agents;

colours;

solvents, solubilizers;

surfactants (emulsifiers, solubilizers, wetting agents, antifoams);

agents affecting the viscosity and consistency, gel formers;

absorption promoters;

adsorbents, humectants, glidants;

agents affecting disintegration and dissolution, fillers (extenders), peptizers;

release-delaying agents.

This list is not definitive; the suitable physiologically acceptable substances are known to the skilled person.

Deoxypeganine can be administered orally or parenterally. It is possible to use known dosage forms such as tablets, coated tablets or pastilles for oral administration. Also suitable are liquid or semiliquid dosage forms, in which case the agent is in the form of a solution or suspension. Solvents or suspending agents which can be used are water, aqueous media or pharmacologically acceptable oils (vegetable or mineral oils). The deoxypeganine-containing medicaments are preferably formulated as depot medicaments which are able to deliver this agent to the body in a controlled manner over a prolonged period.

It is also possible according to the invention for deoxypeganine to be administered by the parenteral route. For this purpose it is particularly advantageous to use transdermal or transmucosal dosage forms for the deoxypeganine administration according to the invention, in particular adhesive transdermal therapeutic systems (agent plasters). These make it possible to deliver the agent in a controlled manner over a prolonged period via the skin to the patient to be treated.

A further advantage is that misuse is less easily possible with parenteral administration forms than with oral dosage forms. The predetermined agent-release area and the predetermined release rate mean that overdosage by the patient can be substantially ruled out. In addition, transdermal dosage forms are very advantageous because of other properties, e.g. avoidance of the first-pass effect or a better, more uniform control of the blood level.

Such transdermal deoxypeganine-containing systems normally have an agent-containing, contact adhesive polymer matrix which is covered on the side remote from the skin by an agent-impermeable backing, and whose adhesive, agent-delivering surface is covered before application by a detachable protective layer. The manufacture of such systems and the basic materials and excipients which can be used therefor are known in principle to the skilled person; for example, the assembly of such transdermal therapeutical systems is described in German patents DE 33 15 272 and DE 38 43 239 or in U.S. Pat. Nos. 4,769,028, 5,089,267, 3,742,951, 3,797,494, 3,996,934 and 4,031,894.

A transdermal therapeutic system (TTS) used according to the invention may have a content of from 0.1 to 50% by weight of deoxypeganine, particularly preferably from 2 to 20% by weight, in the matrix or in the agent reservoir. Suitable deoxypeganine-containing TTS are described, for example, in WO 00/48579.

Also suitable as parenteral dosage forms are solutions for injection, in particular those making a depot effect or delayed and maintained release of the agent possible. Formulations suitable for this purpose are known to the skilled person, e.g. formulations on a nonaqueous basis (e.g. based on physiologically tolerated oils).

It is made possible by the present invention to treat certain concomitant effects or sequelae of chronic abuse of addictive substances, by which means the general wellbeing of these patients is improved and the social reintegration of those chronically harmed by addictive substances is assisted. In addition, the deoxypeganine treatment proposed according to the invention improves the prospects of success of withdrawal therapies and reduces the risk of relapse. The treatment moreover promotes social reintegration of the persons affected.

The described psychiatric or cerebral disturbances, in particular an impairment of cognitive performance or dementia, may also occur as a result of the intake or the abuse of other agents such as environmental poisons (PCB, dioxins, furans, pentachlorophenol, mercury compounds and bromine compounds, amalgam, chlorinated hydrocarbons such as certain solvents), through addictive substances or intoxicants, or as a result of use or abuse of medicines.

Agents therefore mean in principle for the purposes of the invention all psychotropic substances, whether solid, liquid, in vapour or gas form, with which pathological manifestations of the type mentioned occur on single, occasional, frequent or chronic abuse. Besides ethyl alcohol, which has already been mentioned, these agents also include methanol and other alcohols which may be present for example as impurities in alcoholic beverages. The invention relates in particular to the following psychotropic agents and agent-containing preparations, e.g. medicines such as: neuroleptics, antidepressants, tranquilizers (especially benzodiazepines), antipsychotics, hypnotics, psychostimulants (especially amphetamines, “fashionable drugs” such as, for example ecstasy, speed with unstandardized mixtures of agents), further psychotropic drugs and substances, and synthetic derivatives thereof (e.g. based on St John's wort, valerian, hops, melissa, lavender, kava-kava, absinthe; also THC-containing intoxicants such as marihuana and hashish, furthermore cocaine, crack, LSD, psilocybin, mescaline, opium, morphine and morphine derivatives such as heroin, codeine, methadone), wood preservatives such as chromium(VI)-containing wood-preservatives, and certain organic solvents and halogenated carbon compounds or hydrocarbon compounds taken in unintentionally or consumed as intoxicants by “sniffing”, as well as environmental poisons (PCB, dioxins, furans, pentachlorophenol, mercury compounds and bromine compounds, mercury and amalgams.

Even on use as intended of some of the aforementioned substances which are employed for therapeutic purposes it is possible for the side effects mentioned to occur during medically prescribed therapy, especially on repeated or prolonged administration, e.g. cognitive disturbances, cerebral defunctionalization manifestations, psychiatric symptoms, etc.

The present invention therefore provides for the administration of the agent deoxypeganine to treat such disorders which have been caused by the use as intended or abuse of the aforementioned substances. The same applies to intoxication by the environmental poisons mentioned.

It is also known that the side effects mentioned can be caused not only by use or abuse of psychotropic substances but also as a result of single, multiple or chronic administration of other medicaments. The use according to the invention of deoxypeganine therefore also extends to the treatment of symptoms or side effects caused in this way.

Cerebral disturbances or psychiatric symptoms, e.g. memory loss or cognitive disturbances, are also observed in the people affected by acute poisoning (e.g. chemical accidents) and in chronic exposure to poisons (e.g. environmental poisons such as wood preservatives, PCB, dioxins, furans, pentachlorophenol, mercury compounds and bromine compounds, mercury, amalgams, chlorinated hydrocarbons, halogenated biphenyls, tributyltin, wood preservatives, etc.). The use according to the invention of deoxypeganine therefore also extends to the treatment of people who have been adversely affected by exposure to poisons in the aforementioned way.

Finally, the present application also proposes the administration of deoxypeganine in the abovementioned cases to other vertebrates, in particular to mammals, suffering from the symptoms or disturbances described above.

1. Use of deoxypeganine, as free base or as acid addition salt, for the treatment of cerebral, central nervous or psychiatric symptoms, defunctionalization manifestations or diseases occurring through intake of psychotropic substances
as a result of occasional or chronic abuse of addictive substances, intoxicants or medicines, or
as side effect of use, especially repeated or prolonged, as intended of medicaments, or
as an effect of use, especially repeated or prolonged, of medicaments, which use is not the intended use of said medicaments, or
as a result of acute poisoning by psychotropic toxic substances, or
as a result of chronic exposure to psychotropically active toxic substances
in humans and persisting even after successfully completed withdrawal therapy, characterized in that the dose administered is in the range from 0.1 to 100 mg, preferably 10 to 50 mg, per day.

2. Use according to claim 1, characterized in that the symptoms, defunctionalization manifestations or diseases are cognitive disturbances.

3. Use according to claim 1 or 2, characterized in that the abuse of addictive substances is abuse of alcohol.

4. Use according to claim 1 or 2, characterized in that the abuse of addictive substances is the abuse of psychotropic substances, especially of hallucinogenic substances.

5. Use according to any of claims 1 to 4, characterized in that deoxypeganine is used for the treatment of a general loss of control, of memory loss or of an impairment of memory performance, of cognitive ability, of dementia, of perception disturbances, or impairment of muscular coordination.

6. Use according to any of the preceding claims, characterized in that deoxypeganine is administered in a pharmaceutical preparation which contains the agent in proportions of from 0.1 to 90% by weight, preferably 2 to 20% by weight, calculated as free deoxypeganine.

7. Use according to claim 6, characterized in that deoxypeganine is administered in a pharmaceutical preparation which has a depot effect.

8. Use according to claim 6 or 7, characterized in that deoxypeganine is administered orally.

9. Use according to claim 6 or 7, characterized in that deoxypeganine is administered parenterally.

10. Use according to claim 9, characterized in that deoxypeganine is administered transdermally.

11. Use of deoxypeganine, as free base or as acid addition salt, for the manufacture of a medicament for the treatment of cerebral, central nervous or psychiatric symptoms, defunctionalization manifestations or diseases occurring because of intake of psychotropic substances
as a result of occasional or chronic abuse of addictive substances, intoxicants or medicines, or
as side effect of use, especially repeated or prolonged, as intended of medicaments, or
as an effect of use, especially repeated or prolonged, of medicaments, which use is not the intended use of said medicaments, or
as a result of acute poisoning by psychotropic toxic substances, or
as a result of chronic exposure to psychotropically active toxic substances
in humans and persisting even after successfully completed withdrawal therapy, characterized in that the dose to be administered with the said medicament is in the range from 0.1 to 100 mg, preferably 10 to 50 mg, per day.

12. Use according to claim 11, characterized in that the symptoms, defunctionalization manifestations or disorders are cognitive disturbances.

13. Use according to claim 11, characterized in that the abuse of addictive substances is abuse of alcohol.

14. Use according to claim 11, characterized in that the abuse of addictive substances is the abuse of psychotropic substances, in particular of hallucinogenic substances.

15. Use according to any of claims 11 to 14, characterized in that the medicament is used for the treatment of a general loss of control, of memory loss or of impairment of memory performance, of cognitive ability, of dementia, of perception disturbances, or of an impairment of muscular coordination.

16. Use according to any of claims 11 to 15, characterized in that the medicament mentioned contains the agent deoxypeganine in proportions of from 0.1 to 90% by weight, preferably 2 to 20% by weight, calculated as free deoxypeganine.

17. Use according to any of claims 11 to 16, characterized in that the medicament mentioned has a depot effect.

18. Use according to any of claims 11 to 17, characterized in that the medicament mentioned is a medicament which can be administered orally.

19. Use according to any of claims 11 to 17, characterized in that the medicament mentioned is a medicament which can be administered parenterally.

20. Use according to claim 19, characterized in that the medicament mentioned is a medicament which can be administered transdermally.
